(19) United States
(12) Patent Application Publication
(10) Pub. No.: US 2024/0043492 A1
(43) Pub. Date: Feb. 8, 2024

(54) Compositions and Methods of Treating a Coronavirus Infection By Use of an Interferon Fusion Protein

(71) Applicant: East Carolina University, Greenville, NC (US)

(72) Inventor: Mark David Mannie, Greenville, NC (US)

(21) Appl. No.: 17/995,895

(22) PCT Filed: Apr. 26, 2021
(86) PCT No.: PCT/US2021/029067

§ 371 Date: Oct. 10, 2022

Related U.S. Application Data
(60) Provisional application No. 63/015,505, filed on Apr. 25, 2020.

(51) Int. Cl. C07K 14/565; C12N 9/48; A61P 31/14

(52) U.S. Cl. CPC C07K 14/565; C12N 9/485; A61P 31/14; C12Y 304/17023

(57) ABSTRACT

The present inventive concept provides a therapeutic platform that will prevent disease and reverse disease morbidity and mortality wherein the causative agent is a member of a broad class of infectious disease agents that mediate pathogenesis via mechanisms that are ameliorated by an interferon. The platform is based on the construction of single-chain soluble fusion proteins including, for example, a pathogen recognition domain, a linker and an interferon domain.

### CROSS-REFERENCE TO RELATED APPLICATIONS

This application is a 35 § U.S.C. 371 national phase application of PCT International Application No. PCT/US2021/029067, filed Apr. 26, 2021, which claims the benefit of U.S. Provisional Application Ser. No. 63/015,505, filed Apr. 25, 2020, which is incorporated herein by reference in its entirety for its disclosures.

### COPYRIGHT

This disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### STATEMENT REGARDING ELECTRONIC FILING OF A SEQUENCE LISTING

A Sequence Listing in ASCII text format, submitted in accordance with the requirements of 37 C.F.R. §§ 1.821-1.825, entitled 190412-00022_ST25.txt, generated Mar. 16, 2023, 2,352 bytes in size, and filed electronically, is provided in lieu of a paper copy. This Sequence Listing is hereby incorporated herein by reference into the specification for its disclosures.

### FIELD OF THE INVENTION

The present inventive concept relates to compositions and methods of preventing or treating a coronavirus infection by use of a fusion protein-based platform, such as an interferon-based fusion protein platform.

### BACKGROUND

The novel coronavirus (nCoV) Severe Acute Respiratory Syndrome-related Coronavirus 2 or SARS-CoV-2 (referred to as SARS2 herein) is the pandemic virus that originated in November of 2019 in China and has since spread exponentially throughout the global population. SARS2 causes COVID-19 that may culminate in severe and fatal respiratory disease, including Acute Respiratory Distress Syndrome (ARDS). Approximately 5% of patients require intensive care for ARDS, shock, and/or multiorgan failure in association with cytokine-release and vascular-leak mechanisms of immunopathology (1, 2).

### SUMMARY

The present inventive concept provides a therapeutic platform that will prevent disease and reverse disease morbidity and mortality wherein the causative agent is a member of a broad class of infectious disease agents that mediate pathogenesis via mechanisms that are ameliorated by an interferon. The platform is based on the construction of single-chain soluble fusion proteins comprised of a pathogen recognition domain (PRD), a linker and an interferon, and in some aspects of the inventive concept, a PRD, a flexible linker and a human interferon. The order of domains may be N-terminal to C-terminal either PRD-L-IFN or IFN-L-PRD. The pathogen-recognition domain may include pathogen attachment receptors, antibody molecules or fragments, or other molecules that specifically recognize and bind to the pathogen. The linker represents the presence or absence of any sequence of amino acid residues that physically links the pathogen-recognition domain with the interferon domain. The interferon domain may include a Type I (IFN-I), II, or III Interferon, including examples such as an IFN-α, IFN-β, IFN-ω, IFN-κ, IFN-ε, IFN-λ, or IFN-γ. The biologic may be administered prophylactically to prevent infection or therapeutically to reverse ongoing disease progression.

According to an aspect of the inventive concept, provided is a fusion polypeptide including a pathogen recognition domain, a linker region, and an interferon domain, as well as pharmaceutical compositions including the fusion polypeptide, and methods of treating a subject in need thereof and methods of treating a viral infection including administering a therapeutically effective amount of the fusion polypeptide or pharmaceutical composition including the fusion polypeptide to the subject.

According to another aspect of the inventive concept, provided is a fusion polypeptide including an antibody or an antibody fragment domain, a linker region, and an interferon domain, as well as pharmaceutical compositions including the fusion polypeptide, and methods of treating a subject in need thereof and method of treating a viral infection including administering a therapeutically effective amount of the fusion polypeptide or pharmaceutical composition including the fusion polypeptide to the subject.

### BRIEF DESCRIPTION OF THE DRAWINGS

FIG. 1 Depicts examples of recombinant fusion proteins (N-terminal to C-Terminal) of the inventive concept (first three) and recombinant proteins used in development and validation of therapeutic fusion proteins (last six).

FIG. 2 Depicts examples of recombinant fusion proteins (N-terminal to C-terminal) of a modular anti-viral therapeutic platform of the inventive concept wherein the viral recognition/targeting domain is varied for targeting of differing viruses.

### DETAILED DESCRIPTION

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. Additionally, as used herein, the term “and/or” includes any and all combinations of one or more of the associated listed items and may be abbreviated as “/”.

The term “comprise,” as used herein, in addition to its regular meaning, may also include, and, in some embodiments, may specifically refer to the expressions “consist essentially of” and/or “consist of.” Thus, the expression “comprise” can also refer to, in some embodiments, the specifically listed elements of that which is claimed and does not include further elements, as well as embodiments in which the specifically listed elements of that which is claimed may and/or does encompass further elements, or embodiments in which the specifically listed elements of that which is claimed may encompass further elements that do not materially affect the basic and novel characteristic(s) of that which is claimed. For example, that which is claimed, such as a composition, formulation, method, system, etc. “comprising” listed elements also encompasses, for example, a composition, formulation, method, kit, etc. “consisting of,” i.e., wherein that which is claimed does not include further elements, and a composition, formulation, method, kit, etc. “consisting essentially of,” i.e., wherein that which is claimed may include further elements that do not materially affect the basic and novel characteristic(s) of that which is claimed.

The term “about” generally refers to a range of numeric values that one of skill in the art would consider equivalent to the recited numeric value or having the same function or result. For example, “about” may refer to a range that is within ±1%, ±2%, ±5%, ±10%, ±15%, or even ±20% of the indicated value, depending upon the numeric values that one of skill in the art would consider equivalent to the recited numeric value or having the same function or result. Furthermore, in some embodiments, a numeric value modified by the term “about” may also include a numeric value that is “exactly” the recited numeric value. In addition, any numeric value presented without modification will be appreciated to include numeric values “about” the recited numeric value, as well as include “exactly” the recited numeric value. Similarly, the term “substantially” means largely, but not wholly, the same form, manner or degree and the particular element will have a range of configurations as a person of ordinary skill in the art would consider as having the same function or result. When a particular element is expressed as an approximation by use of the term “substantially,” it will be understood that the particular element forms another embodiment.

Unless otherwise defined, all technical and scientific terms used herein have the same meaning as commonly understood by one of ordinary skill in the art to which this invention belongs.

Embodiments of the present inventive concept provide interventions against infectious pathogens that modulate and/or are modulated by the Type I Interferon pathway to mediate disease via mechanisms of direct viral cytopathic activity, excessive inflammatory immunopathogenesis, either alone or together/in combination with any other immunological/virological mechanism(s) of pathogenesis.

Embodiments of the present inventive concept provide a novel therapeutic platform that is designed to prevent disease progression and reverse disease morbidity and mortality of any virus belonging to the Coronaviridae family, for example, but not limited to, MERS CoV, SARS CoV-1, SARS CoV-2 (SARS2), or newly emergent pathogenic serotypes/variants/species of coronavirus, such as SARS CoV-X where X represents any coronavirus that arises in the future, and in an embodiment, prevent disease progression and reverse disease morbidity and mortality of Coronaviridae that cause COVID-19, such as SARS2. The platform is based on the construction of a single-chain soluble fusion polypeptide/protein including: a pathogen recognition domain, such as a truncated soluble ACE2 domain (sACE2), or a fragment thereof; a linker, such as a flexible linker; and an effector/pathogenesis inhibiting domain, for example, an interferon domain, such as a human Interferon-beta (IFN-β) domain, or fragment thereof. The cell-surface transmembrane ACE2 (Angiotensin-Converting Enzyme-2) is the primary viral receptor for SARS2. Accordingly, in embodiments of the inventive concept, the strategy underpinning exemplary construct fusion polypeptides/proteins of the inventive concept is, for example, as follows: (a) The sACE2 domain binds to the Receptor Binding Domain (RBD) of the SARS2 Spike Protein (S protein) and thereby neutralizes the virus and impairs viral infectivity and dissemination. (b) The enzymatic activity of the sACE2 domain compensates for virus-mediated antagonism of transmembrane ACE2 and thereby provides favorable anti-inflammatory activity to counterbalance the pro-inflammatory angiotensin II/AT1R pathway that contributes to the pathogenesis of ARDS. (c) The sACE2 domain anchors an effector that inhibits pathogenesis, such as a Type I Interferon Receptor (IFNAR) agonist, such as IFN-β, to the viral surface and thereby coats the virus with an array of, for example, IFN-β. (d) In exemplary embodiments, an IFN-β fusion protein domain anchored to the surface of the virus triggers signaling through the Type I Interferon Receptor (IFNAR) before infection, such that the virus only infects target cells with upregulated Type I IFN anti-viral defenses. (e) Thus, the virus-targeted effector domain/IFN-β domain drives the IFN-β/CD73/adenosine axis and/or other IFN-mediated anti-inflammatory pathways selectively in virus-infected tissues to reverse vascular permeability and inhibit immunopathogenesis underlying ARDS. (f) The virus-targeted effector domain/IFN-β domain primes any form of cell-mediated memory, such as T cell memory, for example, CD4 and/or CD8 T cell memory, against the SARS2 selectively in tissues bearing high viral (antigen) loads and thereby prevents future reinfection by SARS2 via SARS2-specific cell-mediated immunity, which includes cross-protection against newly emergent serotypes of SARS2. In some embodiments, CD8⁺ T cell memory is primed by the effector domain/IFN-β domain of the fusion polypeptides/proteins of the inventive concept.

According to some embodiments, the physical linkage of the pathogen binding domain, such as sACE2 or fragment thereof and the effector domain/interferon domain, such as IFN-β or fragment thereof, in the form of a single-chain fusion polypeptide/protein, synergizes activities of the domains to provide novel activities that amplify the anti-viral and anti-inflammatory efficacy of the therapy, reflecting mechanisms that would not be fulfilled by either agent alone. For example, the ACE2-IFN-β linkage ensures that the virus only infects cells after IFNAR signaling, thereby obviating virus-mediated antagonism of IFN production (i.e., IFN-β is provided therapeutically and is already replete in the environment). The ACE2-IFN-β linkage also obviates virus-mediated antagonism of IFNAR signaling given that signaling has previously occurred to mobilize IFN-mediated anti-viral defenses before infection. In some embodiments, the pathogen binding domain, for example, a SARS2 viral receptor domain, such as an sACE2 domain, may be directly linked to the interferon domain, for example, an interferon agonist, such as IFN-β. In some embodiments, the SARS2 viral receptor domain, such as an sACE2 domain, may be joined to the interferon agonist, such as IFN-β by any linker that may be appreciated by one of skill in the art. In some embodiments, the linker region/domain may by a polypeptide linker chain/sequence. In some embodiments, the linker may allow the two domains to have sufficient degrees of freedom to interact with their respective ligands regardless of whether the respective ligands are on opposing surfaces or on the same surface, such as a cell surface and/or a virus particle surface, and to move freely relative to one other, i.e., the linker region/domain is a flexible linker. In other embodiments, the linker region/domain may partially or fully constrain movement of the domains relative to one another.

Unlike anti-inflammatory drugs that may be used to treat ARDS, the interferon agonist domain, for example, Type I interferon domains, such as the IFN-β domain of the ACE2-IFN-β fusion protein according to embodiments of the present inventive concept have the potential to reinforce cell-mediated memory, for example, anti-SARS2 T cell memory, such as CD4 and/or CD8 memory responses to provide long-lasting protection against reinfection while blocking over-exuberant pathogenic inflammation. The Interferon domain is also expected to augment humoral antibody-mediated immune responses against the infective virus or viral variant that is present in the body/subject at that time. This fusion protein technology may complement prophylactic vaccines (3) by strengthening cell-mediated memory. In particular, T cell memory, may be advantageous because, for example, CD8 T cells survey conserved internal epitopes not subject to antigenic drift. Virus-specific CD8⁺ T cells and antibody responses are synergistic anti-viral immune defenses, in that antibody blocks viral dissemination, whereas T cell memory responses, such as CD8⁺ T cell responses mediate disease resolution.

An ACE2-IFN-β therapeutic is predicted to drive anti-SARS2 cell-mediated long-term memory that would provide immunity against re-infection by SARS2 and related coronaviruses and/or newly emergent pathogenic serotypes/strains of coronavirus.

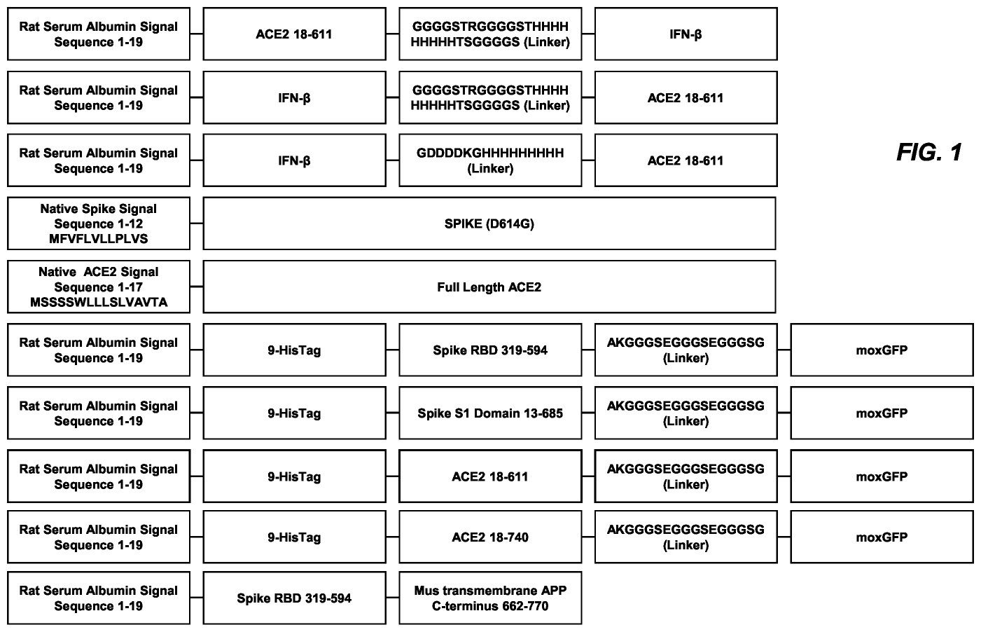

The design of ACE2-IFN-β fusion proteins is guided by structure-function studies that reveal an optimal fusion protein construct. The global design of soluble fusion polypeptides/proteins of the inventive concept include (N-terminal to C-terminal order), for example: ACE2-linker-IFN-β, IFN-β-linker-ACE2, ACE2(R273K)-linker-IFN-β, IFN-linker-β-ACE2(R273K), IFN-β-linker-ACE2(H345L), and ACE2(H345L)-linker-IFN-β proteins, in which ACE2 is enzymatically active, and in which ACE2(R273K) or ACE2(H345L) are enzymatically inactive. The extracellular sACE2 domain (e.g., amino acids 1-740 of NCBI Accession No. NP_001358344.1, amino acids 18-740 without signal peptide) and IFN-β (e.g., NCBI Accession No. NP_002167.1) may include the native N-terminal signal peptide of the N-terminal domain or may include a heterologous signal peptide to optimize expression as a soluble conformationally-intact protein. The sACE2 domain may include different lengths/fragments of ACE2 (4), including, for example, but not limited to: ACE2(aa1-615) (Apeiron APN01); ACE2 aa1-619; ACE2 aa1-611; ACE2 aa1-605; ACE2 aa1-584; ACE2 aa18-619; ACE2: ACE2 aa18-615; aa18-611; ACE2 aa18-605; ACE2 aa18-584, which may represent the natural soluble shed form of the protein (5); and full-length soluble ACE2 aa18-740, or other lengths that may be conducive for expression and activity of the fusion protein. It will be appreciated that full-length soluble ACE2 aa18-740 is dimeric in structure, whereas Apeiron APN01, ACE2 aa18-619, ACE2 aa18-611, ACE2 aa18-605, and ACE2 aa18-584 are monomeric in structure. In some embodiments, the sACE2 domain may be enzymatically/catalytically inactive, for example, the soluble domain from ACE2(R273K) or ACE2(H345L). In some embodiments, the sACE2 domain of the fusion polypeptide/protein is ACE2 aa18-611 (enzymatically active). In some embodiments, the sACE2 domain of the fusion polypeptide/protein is ACE2(H345L) aa18-611 (enzymatically inactive). The linker domain connecting the ACE2 and IFN-β domains in the fusion polypeptides/proteins of the inventive concept is not particularly limited and may vary in length and sequence/amino acid composition based on the results of structure-function optimization studies. For example, in some embodiments, the linker may include the 5-mer G₄S (SEQ ID NO:1), or multimers of the 5-mer set forth in SEQ ID NO:1, for example, a 20-mer including 4 units of the 5-mer set forth in SEQ ID NO:1 (4×G₄S, i.e., GGGGSGGGGSGGGGSGGGGS (SEQ ID NO:2)). In some embodiments, the linker may be a 25-mer including 5 units of the 5-mer set forth in SEQ ID NO:1 (5×G₄S, i.e., GGGGSGGGGSGGGGSGGGGSGGGGS (SEQ ID NO:3). In some embodiments, the linker may be or include the sequence GGGGSTRGGGGSTHHHHHHHHHTSGGGGS (SEQ ID NO:4). In some embodiments, the linker may include the 5-mer DDDDK (SEQ ID NO:5). In some embodiments, the linker may be or include the sequence GDDDDKGHHHHHHHHH (SEQ ID NO:6). In some embodiments, the linker may be or include the sequence AKGGGSEGGGSEGGGSG (SEQ ID NO:7). Exemplary fusion polypeptides/proteins of the inventive concept and fusion polypeptides/proteins for developing and validating fusion polypeptides/proteins of the inventive concept (domains depicted in N-terminal to C-terminal order) are shown in FIG. 1. The first three fusion polypeptides/proteins represent exemplary fusion polypeptides/proteins of the inventive concept, with differing N-terminal/C-terminal domain organization and/or differing linker peptides between domains. The last seven fusion polypeptides/proteins are reagent polypeptides/proteins used in the validation/assessment of fusion polypeptides/proteins of the inventive concept.

In some embodiments of the therapeutic product, the sACE2 virus-targeting domain of the fusion polypeptide/protein is replaced with a monoclonal antibody (mAb) or mAb fragment that is specific for the SARS2 S protein. An example of the mAb fragment would be a single-chain Fv (scFv) fragment that contains the Variable-Light (VL) domain covalently tethered to a Variable-Heavy (VH) domain of an immunoglobulin specific for S. The scFv may be configured (N-terminal to C-terminal) as a VL-VH fragment or a VH-VL fragment. In this application, the scFv mAb domain is specific for nonoverlapping epitopes in the SARS2 S-protein and is used as a viral targeting domain. Anti-S (Spike protein of SARS2) scFv mAbs are fused via an appropriate linker with IFN-β to generate single-chain fusion proteins (i.e., anti-S scFv IFN-β fusion proteins) (scFv-IFN-β). The therapeutic product may include one or multiple scFv-IFN-β fusion proteins with or without the ACE2-IFN-β fusion protein. For example, a “polyclonal” therapeutic may include five different scFv-IFN-β fusion proteins, each with a unique anti-S epitope specificity, in combination with an ACE2-IFN-β therapeutic. These anti-S-IFN-β FPs will provide broad coverage of the SARS2 S protein to neutralize other potential docking sites or proteolytic activation sites that might be important for productive cell infection of SARS2, while increasing the concentration of the IFN-β array displayed on the virus surface. This approach has potential use in synergy with ACE2-IFN-β fusion proteins to enhance anti-viral and anti-inflammatory efficacy in the context of a highly efficacious therapeutic. Notably, scFv fragments are small, easy to express, and are highly suitable for construction of fusion proteins that target viral surface antigens such as the SARS2-S protein. These scFv domains are relatively non-immunogenic and can be humanized by including human framework sequences by gene synthesis.

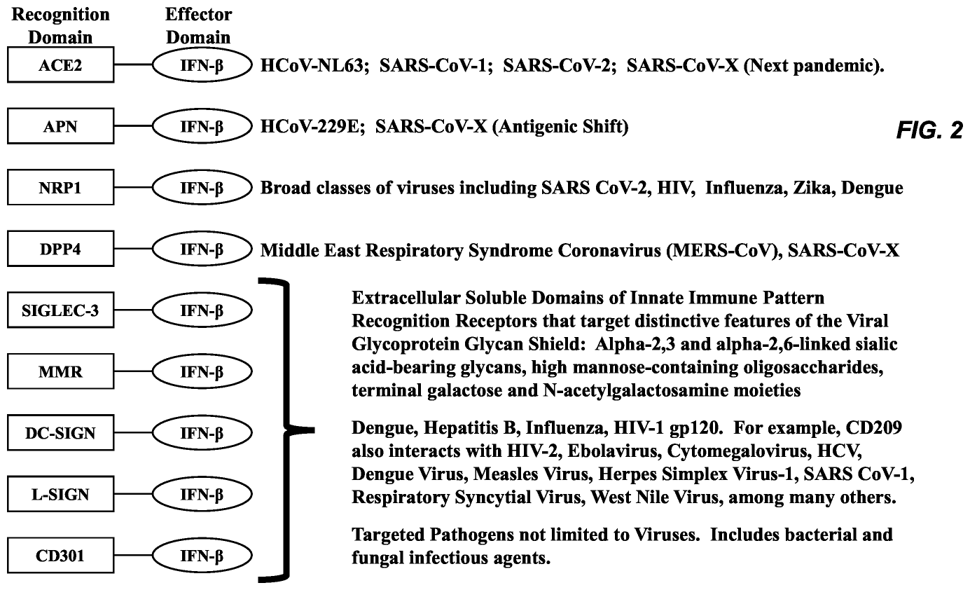

In some embodiments, the sACE2 pathogen recognition domain/virus-targeting domain of the fusion polypeptide/protein may be replaced with an APN, NRP1, DPP4, CD33 (SIGLEC-3), CD329 (SIGLEC-9), CD206 (MMR), CD209 (DC-SIGN), CD299 (L-SIGN), and/or a CD301 domain, including soluble forms thereof. These receptors, in some embodiments, may be used in therapeutics for emerging variants of SARS2 and/or other Coronaviridae family viruses (e.g., HCoV-NL63, HCoV-229E, MERS CoV, SARS CoV-1, SARS CoV-2, emerging SARS CoV (SARS CoV-X) that may result from, for example, antigenic shift, etc.). These receptors, in some embodiments, may be used in therapeutics for Influenza viruses, including emerging variants of Influenza family viruses These receptors, in some embodiments, may be used in therapeutics for alternate viruses, including, for example, HIV, MERS CoV, West Nile Virus and other Flaviviridae family viruses such as Dengue Virus and Zika Virus, among others. For example, NRP1 fusion polypeptides/proteins may target and provide a therapeutic directed toward a broad range of viruses including SARS CoV-2, HIV, Influenza, Zika, and/or Dengue Viruses. DPP4 fusion polypeptides/proteins may target and provide a therapeutic directed toward MERS CoV and/or emerging variants of SARS CoV. CD33 and CD329 bind to specific oligosaccharides on SARS CoV-2 S protein. CD206 interacts with high-mannose oligosaccharides found on many viruses, e.g., Dengue, Hepatitis B, and/or Influenza, and interacts with HIV-1 gp120. CD209 also interacts with HIV-2, Ebolavirus, Cytomegalovirus, HCV, Dengue Virus, Measles Virus, Herpes Simplex Virus-1, SARS CoV-1, Respiratory Syncytial Virus, and/or West Nile Virus, among others. In some embodiments, the pathogen targeted by the fusion polypeptides/proteins of the inventive concept may include bacterial and/or fungal infectious agents. Innate immune receptors, for example, CD33, CD329, CD206, CD209, CD299, and/or CD301, and in some embodiments, those that recognize high-mannose oligosaccharides may be used to target bacterial pathogens such as, but not limited to, for example, Leishmania, Listeria, Helicobacter, Klebsiella, Neisseria, Salmonella, Pseudomonas, Hemophilus, Borrelia, Shigella, Streptococcus, Staphylococcus, Clostridium, and/or Mycobacterium, among others. In some embodiments, innate immune receptors that recognize oligosaccharides may be used to target fungal pathogens such as, but not limited to, for example, Pneumocystis, Cryptococcus, and/or Candida among others. Fusion polypeptide/proteins including alternative pathogen recognition domains/virus-targeting domains and virus targets of the inventive concept are depicted in FIG. 2. In other embodiments of the inventive concept, the interferon domain may be replaced by, for example, an effector domain other than interferon that can inhibit and/or mediate pathogenesis of, for example, any of the viruses as described herein in a subject in need thereof.

The ACE2-IFN-β fusion polypeptides/proteins (as well as the scFv-IFN-β fusion polypeptides/proteins, APN-IFN-β fusion polypeptides/proteins, NRP1-IFN-β fusion polypeptides/proteins, DPP4-IFN-β fusion polypeptides/proteins, CD33-IFN-β fusion polypeptides/proteins, CD329-IFN-β fusion polypeptides/proteins, CD206-IFN-β fusion polypeptides/proteins, CD209-IFN-β fusion polypeptides/proteins, CD299-IFN-β fusion polypeptides/proteins, and/or CD301-IFN-β fusion polypeptides/proteins described herein) in combination with, for example, IL-2 signaling antagonists, and in some embodiments, inhibitors of IL-2 signaling that do not evoke an immune response such as soluble CD25 (sCD25), is a pharmaceutical composition/COVID-19 therapeutic within the scope of the present inventive concept. In some embodiments, an IL-2 signaling antagonist, such as sCD25, inhibits pathogenic IL-2 driven cytokine-storm immunopathogenesis but permits IL-15-driven formation of SARS2-specific CD4⁺ and/or CD8⁺ T cell memory. Soluble CD25 is a natural antagonist of the IL-2 pathway and is comprised of the soluble alpha chain of the high-affinity transmembrane trimeric IL-2αβγ receptor. It has been shown that sCD25 robustly binds and sequesters IL-2 and thereby antagonizes IL-2 signaling while limiting remaining IL-2 to low level concentrations that are conducive to outgrowth of anti-inflammatory CD4⁺ CD25^{high} FOXP3⁺ regulatory T cells (Tregs). In summary, sCD25 is considered advantageous as an adjunct to a COVID-19 therapeutic because sCD25 will block ‘high-zone’ IL-2-mediated immunopathogenesis while promoting low-zone IL-2-mediated anti-inflammatory activity of Tregs in combination with promotion of IL-15 dependent establishment of anti-SARS2 CD8⁺ T cell memory. Exemplary sCD25 polypeptides include, but are not limited to, for example, a recombinant polypeptide including amino acids 1-208 of NCBI Accession No. NP_032393.3 or a recombinant polypeptide including amino acids 1-240 (C213T) of NCBI Accession No. NP_000408.1. In some embodiments, the sCD25 may be a truncated sCD25 polypeptide, for example a recombinant polypeptide including amino acids 1-212 of NCBI Accession No. NP_000408.1. Each of these polypeptides may optionally include a purification sequence, such as a C-terminal 8-histidine sequence, and/or a non-native alanine inserted as the second amino acid to generate an efficient Kozak initiation site for the recombinant sCD25 polypeptides. The combination of the IL-2 signaling antagonist, such as sCD25, and any fusion polypeptide/protein as described herein may be provided to/administered to the subject as a single pharmaceutical composition, i.e., administering of a pharmaceutical composition including a combination of a fusion polypeptide/protein as described herein and an IL-2 signaling antagonist, or the IL-2 signaling antagonist may be administered separately and in addition to fusion polypeptide/protein as described herein without departing from the scope of the inventive concept.

“Polypeptide” as used herein, is used interchangeably with “protein,” and refers to a polymer of amino acids (dipeptide or greater) linked through peptide bonds. Thus, the term “polypeptide” includes proteins, oligopeptides, protein fragments, protein analogs and the like. The term “polypeptide” contemplates polypeptides as defined above that are encoded by nucleic acids, are recombinantly produced, are isolated from an appropriate source, or are synthesized.

As used herein, a “functional” polypeptide is one that retains at least one biological activity normally associated with that polypeptide. Preferably, a “functional” polypeptide retains all of the activities possessed by the unmodified peptide. By “retains” biological activity, it is meant that the polypeptide retains at least about 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits essentially no detectable biological activity normally associated with the polypeptide (e.g., at most, only an insignificant amount, e.g., less than about 10% or even 5%).

“Fusion protein” as used herein, refers to a protein produced when two heterologous nucleotide sequences or fragments thereof coding for two (or more) different polypeptides, or fragments thereof, are fused together in the correct translational reading frame. The two or more different polypeptides, or fragments thereof, include those not found fused together in nature and/or include naturally occurring mutants.

As used herein, a “fragment” is one that substantially retains at least one biological activity normally associated with that protein or polypeptide. In some embodiments, the “fragment” substantially retains all of the activities possessed by the unmodified protein. By “substantially retains” biological activity, it is meant that the protein retains at least about 50%, 60%, 75%, 85%, 90%, 95%, 97%, 98%, 99%, or more, of the biological activity of the native protein (and can even have a higher level of activity than the native protein).

A “recombinant” nucleic acid is one that has been created using genetic engineering techniques.

A “recombinant polypeptide” is one that is produced from a recombinant nucleic acid.

As used herein, an “isolated” nucleic acid (e.g., an “isolated DNA” or an “isolated vector genome”) means a nucleic acid separated or substantially free from at least some of the other components of the naturally occurring organism or virus, such as for example, the cell or viral structural components or other polypeptides or nucleic acids commonly found associated with the nucleic acid.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide. As used herein, the “isolated” polypeptide is at least about 25%, 50%, 60%, 70%, 75%, 80%, 85%, 90%, 95%, 97%, 98%, 99% or more pure (w/w).

The term “prevent,” “preventing” or “prevention of” (and grammatical variations thereof) refers to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the inventive concept. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression are less than what would occur in the absence of carrying out the steps of the methods of the present invention.

As used herein, the expression “treat,” “treating” “treatment of” (and grammatical variations thereof) infection means improving, reducing, or alleviating at least one symptom or biological consequence of the infection in a subject, and/or reducing or decreasing virus titer, load, replication or proliferation in a subject following exposure to the virus. The expression “treating infection” also includes shortening the time period during which a subject exhibits at least one symptom or biological consequence of virus infection after being infected by a virus. The subject may exhibit or be diagnosed with one or more symptoms or biological consequences of virus infection.

A “therapeutically effective amount,” “treatment effective amount” and “effective amount” as used herein are synonymous unless otherwise indicated, and mean an amount of a composition or formulation of the present inventive concept that is sufficient to improve the condition, disease, or disorder being treated and/or achieved the desired benefit or goals as described herein. Those skilled in the art will appreciate that the therapeutic effects need not be complete or curative, as long as some benefit is provided to the subject. Similarly, a “prevention effective” amount is an amount that is sufficient to prevent (as defined herein) the disease, disorder and/or clinical symptom in the subject. Those skilled in the art will appreciate that the level of prevention need not be complete, as long as some benefit is provided to the subject.

In some embodiments, the virus prevented or treated according to the present inventive concept is any virus belonging to the Coronaviridae family now known or yet to be discovered. Generally, there are four main sub-groupings of coronaviruses, known as alpha, beta, gamma, and delta. Exemplary coronaviruses include 229E (alpha coronavirus), NL63 (alpha coronavirus), OC43 (beta coronavirus), HKU1 (beta coronavirus), MERS-CoV (the beta coronavirus associated with Middle East Respiratory Syndrome, or MERS), SARS-CoV (the beta coronavirus associated with severe acute respiratory syndrome, or SARS) and SARS-CoV-2 (the novel coronavirus associated with coronavirus disease 2019, or COVID-19).

“Subject” as used herein may be a patient. In some embodiments, the subject is a human; however, a subject of this disclosure can include an animal subject, particularly mammalian subjects such as canines, felines, bovines, caprines, equines, ovines, porcines, rodents (e.g. rats and mice), lagomorphs, primates (including non-human primates), etc., including domesticated animals, companion animals and wild animals for veterinary medicine, treatment or pharmaceutical drug development purposes.

The subjects relevant to this disclosure may be male or female and may be any species and of any race or ethnicity, including, but not limited to, Caucasian, African-American, African, Asian, Hispanic, Indian, etc., and combined backgrounds. The subjects may be of any age, including newborn, neonate, infant, child, adolescent, adult, and geriatric.

In some embodiments, the subject is at high risk for contracting a coronavirus. In some embodiments, the subject is aged 65 or older, has high blood pressure, asthma, lung disease, cancer, diabetes, Down syndrome, heart disease/conditions, HIV, kidney disease, liver disease, lung disease, sickle cell disease or thalassemia, a neurological condition such as dementia, a substance use disorder, had a solid organ or blood stem cell transplant, and/or had a stroke/cerebrovascular disease, is pregnant, is overweight/obese, smokes, and/or is immunocompromised. In some embodiments, the immunocompromised subject may have an immunodeficiency disease and/or may have a deficiency in Type I IFN defenses.

In terms of administration, the most suitable route (parenteral, oral, nasal, inhalational, ocular, transmucosal and transdermal) in any given case will depend on the nature and severity of the condition being treated and on the fusion protein, viral vector, nucleic acid and/or pharmaceutical formulation being administered. In some embodiments, routes of administration are parenteral injection such as intravenous, subcutaneous, intramuscular, intradermal, etc.)

### BIBLIOGRAPHY & REFERENCES CITED

1. Rodriguez-Morales A J, Cardona-Ospina J A, Gutierrez-Ocampo E, Villamizar-Pena R, Holguin-Rivera Y, Escalera-Antezana J P, et al. Clinical, laboratory and imaging features of COVID-19: A systematic review and meta-analysis. Travel Med Infect Dis (2020):101623. Epub 2020/03/18. doi: 10.1016/j.tmaid.2020.101623. PubMed PMID: 32179124.
2. Harcourt J, Tamin A, Lu X, Kamili S, Sakthivel S K, Murray J, et al. Severe Acute Respiratory Syndrome Coronavirus 2 from Patient with 2019 Novel Coronavirus Disease, United States. Emerg Infect Dis (2020) 26(6). Epub 2020/03/12. doi: 10.3201/eid2606.200516. PubMed PMID: 32160149.
3. Walls A C, Park Y J, Tortorici M A, Wall A, McGuire A T, Veesler D. Structure, Function, and Antigenicity of the SARS-CoV-2 Spike Glycoprotein. Cell (2020). Epub 2020/03/11. doi: 10.1016/j.cell.2020.02.058. PubMed PMID: 32155444.
4. Wysocki J, Schulze A, Bathe D. Novel Variants of Angiotensin Converting Enzyme-2 of Shorter Molecular Size to Target the Kidney Renin Angiotensin System. Biomolecules (2019) 9(12). Epub 2019/12/22. doi: 10.3390/biom9120886. PubMed PMID: 31861139; PubMed Central PMCID: PMCPMC6995632.
5. Jia H P, Look D C, Tan P, Shi L, Hickey M, Gakhar L, et al. Ectodomain shedding of angiotensin converting enzyme 2 in human airway epithelia. Am J Physiol Lung Cell Mol Physiol (2009) 297(1):L84-96. Epub 2009/05/05. doi: 10.115/ajplung.00071.2009. PubMed PMID: 19411314; PubMed Central PMCID: PMCPMC2711803.

Having described various aspects of the present invention, the same will be explained in further detail in the following examples, which are included herein for illustration purposes only, and which are not intended to be limiting to the invention.

### EXAMPLE 1

### Construction and Validation of ACE2-IFN-β Fusion Proteins (FPs)

This example focuses on expression, purification, and characterization of the ACE2-IFN-β FPs together with assays validating domain structure and function (Table 1). This example is designed to provide an optimal COVID-19 therapeutic protein that can be advanced in a clinical development program. This example requires novel expression systems for the FPs plus novel GFP or DsRed-tagged soluble Spike proteins or ACE2 proteins and validates activities of the FP-ACE2 and FP-IFN-β domains (Table 2). For secreted recombinant proteins (Table 2, rows 1-6, 10-15), the rat serum albumin signal sequence is used to direct the expressed protein to the ER/golgi secretory pathway (1). Soluble proteins include an 8×Histag and 2×Strep-Tags. Soluble human ACE2 proteins include the monomeric 18-611 (1a-5a, 13a-b) and the dimeric 18-740 domain (1b-5b, 14a-b), the latter of which includes the collectrin domain that confers the ACE2 dimeric structure (2, 3). The GS linker (rows 1-2) includes a 20-mer 4×G₄S (SEQ ID NO:2) sequence, which is a common strategy in linking domains in single-chain FPs. The GDDDDKG (SEQ ID NO:8) linker (row 3) was previously used to express a functionally-intact IFN-β domain in tolerogenic IFN-β-neuroantigen FPs (4-6). The H345L mutation (row 4) abrogates ACE2 enzymatic activity without disrupting structural stability (3). The human IgG1-Fc domain (row 5) is used to confer stability to the FP (3). Full-length SARS2 Spike is a transmembrane trimeric structure and bears the D614G mutation (row 7a) that was implicated in increased viral spread. Full-length ACE2 is a transmembrane dimeric structure (row 7b). The transmembrane APP662-770 FPs (row 8) represent the RBD or sACE2 domains fused to the transmembrane/cytoplasmic domains of Amyloid Precursor Protein membrane anchor sequence. These APP-fusions are monomeric, transmembrane proteins. The stabilized Spike protein (SARS CoV-2 spike Hexapro variant) (row 12) (7) includes 6 proline substitutions and a null GSAS mutation of the S1/S2 furin cleavage site. These mutations increase expression due to augmented protein stability. ACE2 and IFN-β of these experiments are human sequences.

TABLE 1
Experimental approach

| Focus | Manipulation | Measurements | Outcome |
|---|---|---|---|
| a: Validation of IFN-β domain | FPs vs IFN-β | IFN-β specific | FP-IFNβ domain |
| | | assays | equipotent vs IFN-β |
| b: Validation of ACE2 domain | FPs vs sACE2 | Binding of ACE2 | FP-ACE2 domain |
| | | domain to Spike | equipotent vs sACE2 |
| | | protein domains | |
| c: Synergy of ACE2-IFN-β | FPs vs IFN-β | Inhibition of | FP >> IFN-β for |
| linkage | | proliferation of | inhibition of Spike- |
| | | Spike-transfectants | transfectants |
| d: Inhibition of fusion & syncytia | FPs vs sACE2 | Block fusion of | FP-ACE2 domain |
| | | Spike-HEK and | equipotent vs sACE2 |
| | | ACE2-HEK | |
| e: Inhibition of SARS2 | FPs vs ACE2 vs | Inhibition of SARS2 | FP >> IFN-β or ACE2 |
| replication | IFN-β | viral replication | or non-linked |
| | | | combination |

TABLE 2
Recombinant proteins for Example 1

| | (a) Monomeric | (b) Dimeric |
|---|---|---|
| | Therapeutic Proteins | Therapeutic Proteins |
| 1 | sACE2(18-611)-GS-IFNβ | sACE2(18-740)-GS-IFNβ |
| 2 | IFNβ-GS-sACE2(18-611) | IFNβ-GS-sACE2(18-740) |
| 3 | IFNβ-DDDDK-sACE2 (18-611) | IFNβ-DDDDK-sACE2 (18-740) |
| 4 | IFNβ-Linker-sACE2 (18-611) (H345L) | IFNβ-Linker-sACE2 (18-740) (H345L) |
| 5 | IFNβ-Linker-sACE2 (18-611) IgG1-Fc | IFNβ-Linker-sACE2 (18-740) IgG1-Fc |
| 6 | IFN-β | IFN-β-Linker-IgG1-Fc |
| | (a) Transmembrane | (b) Transmembrane |
| | Spike Protein | ACE2 Protein |
| 7 | Full-Length Spike D614G | Full Length ACE2 |
| 8 | Spike RBD_APP662-770 | ACE2(18-611)_APP662-770 |
| | (a) Monomeric | (b) Monomeric |
| | GFP-Conjugates | DsRed Conjugates |
| 10 | Soluble RBD-moxGFP | DsRed-Soluble RBD |
| 11 | Soluble S1-moxGFP | DsRed-Soluble S1 |
| 12 | Soluble Stabilized-Spike-moxGFP | DsRed-Soluble Stabilized-Spike |
| 13 | Soluble ACE2(18-611)-moxGFP | DsRed-Soluble ACE2(18-611) |
| 14 | Soluble ACE2(18-740)-moxGFP | DsRed-Soluble ACE2(18-740) |
| 15 | Soluble ACE2(18-611) | Soluble ACE2(18-740) |

The constructs contain 20 amino acid (4×G₄S, SEQ ID NO:2) (GS, Table 2) linkers to maximize flexibility and non-interference of the ACE2 and IFN-β domains. Long flexible linkers are preferred whereby the ACE2 and IFN-β domains have sufficient degrees of freedom to interact with their respective ligands regardless of whether Spike and IFNAR are on opposing surfaces or are on the same cell surface. Herein, the ACE2 domain is free to interact with the viral surface whereas the IFN-β domain has the freedom to interact with IFNAR on adjacent cells before potential infection. Conversely, the ACE2 domain has the freedom to interact with Spike proteins on the surface of infected cells (which escape from the ER-golgi compartment) and the IFN-β domain has the freedom to interact with IFNAR on the same cell surface. In the latter case, IFNAR signaling in virally-infected host cells may drive apoptosis before full viral maturation to limit viral dissemination. We have extensive experience in successfully constructing IFN-β-neuroantigen FPs with fully functional subdomains (4-6, 8). We have a validated stable CHO line for expression of human IFN-β-8his and have shown that the C-term terminal 8-histag does not impair IFN-β expression or activity. Proteins are expressed in HEK or CHO expression systems. HEK cells are advantageous because HEK cells provide native human patterns of glycosylation. Alternatively, because human IFN-β may have anti-proliferative activity on HEK cells, and whereas human IFN-β lacks biological activity on CHO cells (i.e., human IFN-β lacks cross-reactivity with hamster IFNAR), transfections may be conducted with both HEK and CHO cells to optimize the stable expression system. Proteins may be purified on Ni-NTA columns via the C-terminal 8-histag and analyzed for yield (optical density), purity (SDS-PAGE), and oligomeric state (analytic gel filtration). Additional combinatorial FPs may be made, depending on results.

Both active ACE2 and inactive ACE2(H345L) (3) are tested to determine whether ACE2 enzymatic activity is advantageous for a COVID-19 therapeutic. The literature supports a rationale that the active soluble version may have a favorable activity profile in ARDS. ACE2 cleaves many peptide substrates and is known to mediate the cleavage of angiotensin II to angiotensin (1-7) to mediate vasodilatation and drive the anti-inflammatory ACE2/Ang1-7/Mas1R pathway while antagonizing the pro-inflammatory angiotensin II/AT1R/NFκB pathway (9-13). ACE2 also mediates a protective role in acute lung injury. For example, active ACE2 but not the mutant inactive recombinant ACE2 protein alleviated acute lung injury in wild-type and ACE2^{-/-} mice (14). ACE2 is antagonized by SARS-CoV in that ACE2 surface expression and mRNA expression on the apical surface of differentiated ciliated airway epithelia cells is downregulated upon interaction with SARS-CoV or recombinant S protein. ACE2 not only inhibits viral entry into target cells (14), but active ACE2 may also compensate for SARS2-mediated antagonism of endogenous ACE2 by blocking viral polarization of the pro-inflammatory angiotensin II system. Conversely, a recombinant therapeutic that is an active protease may have unpredicted adverse effects on FP stability. The decision point on whether an active or inactive ACE2 domain is preferred in our studies is determined by outcomes in this example and the outcomes of the Apeiron clinical trial, which, if successful, supports use of the active ACE2 domain.

This example focuses on expression, purification, and characterization of the FPs incorporating sACE2 and IFN-β domains together with assays to validate domain structure and function. The main question focuses on the optimal structure for a FP incorporating sACE2 and IFN-β domains in reference to N-terminal to C-terminal orientation and the linker sequences. Optimal expression levels and protein stability are assessed by assaying function of the individual domains and synergistic functions due to the linkage of the two domains.

### a: Validation of the IFN-β Domain

The biological activity of the IFN-β FP domain is assessed by comparing the FPs to monomeric IFN-β across a concentration range of 1 pM to 1 μM in a series of IFN-β-specific assays, including anti-proliferative activity and induction of pSTAT1, and induction of FOXP3 Tregs in human PBMC cultures (6). To measure anti-proliferative activity, human PBMCs (commercial, de-identified samples) are stimulated with GM-CSF (myeloid responses), Con-A/IL-2 (T cell responses), or IL-4 & anti-IgM F(ab′)₂ (B cells). Human lymphoid and myeloid cancer cell lines may also be suitable for these assays. IFN-β is expected to inhibit [³H]thymidine incorporation in all of these culture systems. Potency of the FP-IFN-β domains is compared with IFN-β for induction of pSTAT1 in flow cytometric assays, which are standard in the laboratory. Lastly, based on published data (6), IFN-β directly increases percentages and numbers of CD4⁺ CD25^{high} FOXP3⁺ T cells in mitogen-stimulated cultures of purified naïve CD4⁺ T cells and irradiated myeloid DCs. Tests for equipotency are performed for the FP-IFN-β domains versus IFN-62 in assays measuring induction of FOXP3. To complement these assays, HEK-Blue™ IFN-α/β cells (InVivogen) are used to quantitatively compare FP-IFN-β and IFN-β in assays measuring activation of the ISGF3 pathway. If the IFN-β domain of the FPs have potency and efficacy profiles equal to those of monomeric IFN-β, the IFN-β domain is fully functionally as a domain of the FP. If not, the linker between the ACE2 domain and the IFN-β domain is re-engineered to optimize flexibility and physical independence of the domains. The FP-IFN-β domain is assessed for potency compared to the free soluble monomeric IFN-β depending on whether the FP-IFN-β domain is expressed in the context of dimeric ACE2 (rows 1-5) with or without stabilization by human IgG1 Fc domain.

### b: Validation of the ACE2 Domain

Structural integrity of FP-ACE2 domains is assessed by comparing FPs with soluble ACE2 proteins (Table 2, rows 13-15) in binding assays measuring the interaction of the FP-ACE2 domains or soluble ACE2 proteins with the SARS2 Spike protein. HEK or CHO cells stably transfected with SARS2 Spike transmembrane protein (e.g., HEK-S or CHO-S cells) are used. The FPs and ACE2 control proteins are incubated with HEK-S cells or non-transfected control cells over a concentration range of 1 pM to 10 μM. Because all recombinant proteins have a C-terminal 8-histag, surface binding to HEK-S cells are detected with an fluorochrome-labeled anti-Histag secondary Ab together with the moxGFP or DsRed tag by flow cytometry. Affinity, including association/dissociation rate constants, for the interaction of the FP-ACE2 domains and control ACE2 proteins with the soluble receptor binding domain (RBD) of the SARS2 Spike protein are measured by surface plasmon resonance (SPR). If the FP-ACE2 domain interacts with the SARS2 Spike protein and the respective receptor binding domain with profiles similar to that of soluble ACE2, the ACE2 domain is fully functional as a domain of the FP. If not, the ACE2 domain is re-engineered to test alternative fragments such as ACE2 aa18-584 (15, 16) or the linker domains are re-engineered. Enzymatic activity is assessed with, for example, a Fluorometric ACE2 Activity Assay Kit (BioVision). Assessment whether dimeric versions of sACE2 have superior activities compared to monomeric ACE2, and whether stabilization with the human IgG1 domain confers more potent activity is examined. The experimental design reveals whether inactivation of sACE2 enzymatic (the H345L mutation) (3) confers advantages or disadvantages in regard to expression, stability, or activity. The outcome is an expression system for a FP that incorporates functionally-intact ACE2 and IFN-δ domains.

### c: Synergy of ACE2-IFN-β Linkage

ACE2-mediated, IFN-β-targeting activity of the FPs is assessed by comparing FPs to IFN-β across a concentration range of 1 fM to 1 μM for anti-proliferative activity in cell lines transfected or not with transmembrane Spike protein, including the TF1 cell line (e.g. TF1-S versus non-transfected TF1 lines). TF1 cells are particularly susceptible to the cytotoxic action of IFN-β. The FPs, by targeting IFN-β via the sACE2/Spike protein interaction onto the cell surface of transfected cell lines, engenders enhanced IFNAR signaling and exhibit left-shifted anti-proliferative curves compared to IFN-β alone. That is, FPs have enhanced anti-proliferative potency compared to IFN-β, and the magnitude of the left-shift should be proportional to the targeting of IFN-β to IFN-β-susceptible Spike⁺ lines. Conversely, FPs do not elicit left-shifted kill curves in non-transfected cells. These data directly assess the linker sequence in providing synergic, ACE2-mediated targeting of IFN-β to cell surfaces bearing transmembrane Spike protein. These analyses complement those described in sections a: and b: above validating the IFN-β and ACE2 domains in determining whether ACE2 enzymatic activity, ACE2 dimerization, or IgG1-Fc domains facilitate IFN-β-mediated anti-proliferative activity and cell killing when IFN-β is targeted to cell membranes by the ACE2-Spike interaction.

### d: FP Inhibition of Cell Fusion and Formation of Cell-to-Cell Syncytia

A surrogate assay of membrane fusion is utilized to assess whether FPs, compared to sACE2 more efficiently inhibit Spike-mediated membrane fusion. HEK cell lines, transfected and untransfected, with the gene encoding the full-length transmembrane Spike(D614G) (i.e., HEK-S cells) or full-length transmembrane ACE2 (HEK-ACE2) are derived. HEK-S and HEK-ACE2 cells are differentially labeled with cell tracking dyes (i.e., cytoplasmic dyes or lipophilic cell-trace dyes) and then mixed together in culture. The HEK-S and HEK-ACE2 cells fuse to form syncytia (17, 18), which are detected by mixing of the cell-tracking dyes via fluorescence microscopy and flow cytometry. This assay may require addition/transfection of proteases (e.g., TMPRSS2) to activate the fusion machinery of the Spike protein. The FPs, soluble ACE2 proteins, and IFN-p are compared over a concentration range of 1 pM to 1 μM to assess prevention of fusion of HEK-S and HEK-ACE2 cells. The soluble FP-ACE2 domain is predicted to bind HEK-S cells and neutralize the Spike protein to prevent HEK-ACE2 cell interaction with HEK-S, thereby preventing cell fusion, with a Kᵢ equivalent to that of sACE2.

### e: FP-Mediated Neutralization of SARS2 Replication In Vitro in VeroE6 Cells

A 2019-nCoV SARS CoV-2 clinical isolate is obtained, for example, from BEI Resources Repository, NIH. The FPs is compared to sACE2, IFN-β, the unlinked combination of sACE2 and IFN-β, or no protein as the major variables (5 major groups). These reagents are tested for inhibitory activity in assays measuring the replication of SARS2 in VeroE6 culture systems. These reagents are incubated with either virus or cells before mixing of virus with cells. More specifically, VeroE6 cells are plated overnight in 96-well plates. FPs or control proteins are incubated with the SARS2 virus for 1 hr to allow binding of the FPs or control proteins to the viral surface before addition of the mixture to the cell culture, and the mixture is incubated with the cells from 0.032 to 3.2 MOI (half-log increments) for 1 hr. The cells are washed to remove free virus and then incubated for 18-24 hrs. Cell lysates are collected for analysis by qPCR via a CFX96 Touch Real-Time PCR Detection System (Bio-Rad). In addition, VeroE6 cultures are pre-incubated with a concentration range (1 pM to 1 μM) of FPs versus control proteins before addition of virus. Together, these experiments test whether the covalent IFN-β-ACE2 linkage synergistically potentiates IFN-β-mediated anti-viral activity compared to either activity alone. By comparing unimpeded virus, ACE2-neutralized virus, and ACE2-neutralized virus presenting a surface array of IFN-β, these experiments show that IFN-β, targeted to the viral surface, abrogates viral infectivity. The broader implication is that IFN-β exposure pre-infection interferes with viral replication, whereas IFN-β exposure post-infection has a more limited anti-viral efficacy.

Overall, this example provides a comprehensive assessment of the FP platform, including the functional integrity of the IFN-β and ACE2 domains, the IFN-β targeting functionality of the linker domain, and FP-mediated neutralization of ACE2/S-mediated membrane fusion. This example provides a strong foundation to assess the ability of these FPs neutralize live SARS2 while coating the viral surface with an array of IFN-β. Most importantly, this example provides the foundation for development of promising FPs as a novel therapeutic for COVID-19.

### Bibliography & References Cited, Example 1

1. Wilkinson D S, Ghosh D, Nickle R A, Moorman C D, Mannie M D. Partial CD25 antagonism enables dominance of antigen-inducible CD25high FOXP3+ regulatory T cells as a basis for a Treg-based adoptive immunotherapy. Front Immunol (2017) https://doi.org/10.3389/fimmu.2017.01782. PubMed PMID: 29312311; PubMed Central PMCID: PMC5735073.
2. Yan R, Zhang Y, Li Y, Xia L, Guo Y, Zhou Q. Structural basis for the recognition of SARS-CoV-2 by full-length human ACE2. Science (2020) 367(6485):1444-8. Epub 2020/03/07. doi: 10.1126/science.abb2762. PubMed PMID: 32132184.
3. Glasgow A, Glasgow J, Limonta D, Solomon P, Lui I, Zhang Y, Nix M A, Rettko N J, Zha S, Yamin R, Kao K, Rosenberg O S, Ravetch J V, Wiita A P, Leung K K, Lim S A, Zhou X X, Hobman T C, Kortemme T, Wells J A. Engineered ACE2 receptor traps potently neutralize SARS-CoV-2. Proc Natl Acad Sci USA (2020) 117(45):28046-55. Epub 2020/10/24. doi: 10.1073/pnas.2016093117. PubMed PMID: 33093202; PubMed Central PMCID: PMCPMC7668070.
4. Mannie M D, Abbott D J, Blanchfield J L. Experimental autoimmune encephalomyelitis in Lewis rats: IFN-beta acts as a tolerogenic adjuvant for induction of neuroantigen-dependent tolerance. J Immunol. (2009) 182(9):5331-41. Epub 2009/04/22. doi: 10.4049/jimmunol.0803756. PubMed PMID: 19380780.
5. Mannie M D, Blanchfield J L, Islam S M, Abbott D J. Cytokine-neuroantigen fusion proteins as a new class of tolerogenic, therapeutic vaccines for treatment of inflammatory demyelinating disease in rodent models of multiple sclerosis. Front Immunol (2012) 3:255. Epub 2012/08/31. doi: 10.3389/fimmu.2012.00255. PubMed PMID: 22934095; PubMed Central PMCID: PMCPMC3422719.
6. Wang D, Ghosh D, Islam S M, Moorman C D, Thomason A E, Wilkinson D S, Mannie M D. IFN-beta Facilitates Neuroantigen-Dependent Induction of CD25+ FOXP3+ Regulatory T Cells That Suppress Experimental Autoimmune Encephalomyelitis. J Immunol (2016) 197:2992-3007. Epub 2016/09/14. doi: 10.4049/jimmunol.1500411. PubMed PMID: 27619998; PubMed Central PMCID: PMC5101178.
7. Hsieh C L, Goldsmith J A, Schaub J M, DiVenere A M, Kuo H C, Javanmardi K, Le K C, Wrapp D, Lee A G, Liu Y, Chou C W, Byrne P O, Hjorth C K, Johnson N V, Ludes-Meyers J, Nguyen A W, Park J, Wang N, Amengor D, Lavinder J J, Ippolito G C, Maynard J A, Finkelstein I J, McLellan J S. Structure-based design of prefusion-stabilized SARS-CoV-2 spikes. Science (2020) 369(6510):1501-5. Epub 2020/07/25. doi: 10.1126/science.abd0826. PubMed PMID: 32703906; PubMed Central PMCID: PMCPMC7402631.
8. Mannie M D, Curtis A D, 2nd. Tolerogenic vaccines for Multiple sclerosis. Hum Vaccin Immunother (2013) 9(5):1032-8. Epub 2013/01/30. PubMed PMID: 23357858.
9. Calo L A, Rigato M, Bertoldi G. ACE2/Angiotensin 1-7 protective antiinflammatory and antioxidant role in hyperoxic lung injury: support from studies in Bartter's and Gitelman's syndromes. QJM (2019). Epub 2019/12/19. doi: 10.1093/qjmed/hcz319. PubMed PMID: 31851364.
10. Cheng H, Wang Y, Wang G Q. Organ-protective Effect of Angiotensin-converting Enzyme 2 and its Effect on the Prognosis of COVID-19. J Med Virol (2020). Epub 2020/03/30. doi: 10.1002/jmv.25785. PubMed PMID: 32221983.
11. Fang Y, Gao F, Liu Z. Angiotensin-converting enzyme 2 attenuates inflammatory response and oxidative stress in hyperoxic lung injury by regulating NF-kappaB and Nrf2 pathways. QJM (2019) 112(12):914-24. Epub 2019/08/09. doi: 10.1093/qjmed/hcz206. PubMed PMID: 31393582.
12. Wu Y. Compensation of ACE2 Function for Possible Clinical Management of 2019-nCoV-Induced Acute Lung Injury. Virol Sin (2020). Epub 2020/02/09. doi: 10.1007/s12250-020-00205-6. PubMed PMID: 32034638.
13. Ye R, Liu Z. ACE2 exhibits protective effects against LPS-induced acute lung injury in mice by inhibiting the LPS-TLR4 pathway. Exp Mol Pathol (2020) 113:104350. Epub 2019/12/06. doi: 10.1016/j.yexmp.2019.104350. PubMed PMID: 31805278.
14. Jia H. Pulmonary Angiotensin-Converting Enzyme 2 (ACE2) and Inflammatory Lung Disease. Shock (2016) 46(3):239-48. Epub 2016/04/16. doi: 10.1097/shk.0000000000000633. PubMed PMID: 27082314.
15. Wysocki J, Schulze A, Bathe D. Novel Variants of Angiotensin Converting Enzyme-2 of Shorter Molecular Size to Target the Kidney Renin Angiotensin System. Biomolecules (2019) 9(12). Epub 2019/12/22. doi: 10.3390/biom9120886. PubMed PMID: 31861139; PubMed Central PMCID: PMCPMC6995632.
16. Jia H P, Look D C, Tan P, Shi L, Hickey M, Gakhar L, Chappell M C, Wohlford-Lenane C, McCray P B, Jr. Ectodomain shedding of angiotensin converting enzyme 2 in human airway epithelia. Am J Physiol Lung Cell Mol Physiol (2009) 297(1):L84-96. Epub 2009/05/05. doi: 10.1152/ajplung.00071.2009. PubMed PMID: 19411314; PubMed Central PMCID: PMCPMC2711803.
17. Buchrieser J, Dufloo J, Hubert M, Monel B, Planas D, Rajah M M, Planchais C, Porrot F, Guivel-Benhassine F, Van der Werf S, Casartelli N, Mouquet H, Bruel T, Schwartz O. Syncytia formation by SARS-CoV-2-infected cells. EMBO J (2020) 39(23):e106267. Epub 2020/10/15. doi: 10.15252/embj.2020106267. PubMed PMID: 33051876; PubMed Central PMCID: PMCPMC7646020.
18. Thakur N, Conceicao C, Isaacs A, Human S, Modhiran N, McLean R K, Pedrera M, Tan T K, Rijal P, Townsend A, Taylor G, Young P R, Watterson D, Chappell K J, Graham S P, Bailey D. Micro-fusion inhibition tests: quantifying antibody neutralization of virus-mediated cell-cell fusion. J Gen Virol (2020). Epub 2020/10/16. doi: 10.1099/jgv.0.001506. PubMed PMID: 33054904.

### EXAMPLE 2

### Differential Usage of Accessory Host Receptors by Emerging SARS CoV-2 Variants

First (a.), this example describes assessment of the binding of alternative host receptors (Table 3) to the Spike domains on infectious variants including the B (14), B.1.5 (15-19), B.1.1.7 (1-4), B.1.351 (5-10), P.1 (10-13), B.1.429 (8), and B.1.526 (20) lineages in comparison to other control viruses. The example also assesses the binding of these host receptors to Spike proteins expressed by stably-transfected HEK cells, including Spike proteins from SARS-CoV-1, MERS, HCoV-229E, and HCoV-OC43. Second (b.), the project uses commercially available fluorochrome-conjugated mAbs specific for these host receptors (Table 3) to map expression of these host transmembrane receptors on nascent and differentiated lineages of human leukocytes. Third (c.), the project will assess whether selected SARS CoV-2 variants infect or dock onto leukocytic subsets or differentiated leukocytic lines that express high levels of particular host receptors. Overall, this example defines variant Spike/host receptor/leukocyte subset axes that may underpin COVID-19.

TABLE 3
Potential receptors and/or docking sites for SARS CoV-2.

| | | Alternative | Extracellular |
|---|---|---|---|
| | Name | name | domain |
| 1 | sACE2 | Angiotensin Converting Enzyme-2 | sACE2 aa18-740 |
| 2 | sAPN | Aminopeptidase CD13 | sAPN aa33-967 |
| 3 | sNRP1 | Neuropilin-1/CD304 | sNRP1 aa22-856 |
| 4 | sDPP4 | Dipeptidyl-Peptidase-4/CD26 | sDPP4 aa29-766 |
| 5 | sCD33 | Siglec-3 | sCD33 aa18-259 |
| 6 | sCD329 | Siglec-9 | sCD329 aa18-348 |
| 7 | sCD206 | Macrophage Mannose Receptor | sCD206 aa655-1213 |
| 8 | sCD209 | DC-SIGN/CLEC4L | sCD209 aa59-404 |
| 9 | sCD299 | CLEC4M/CD209L/L-SIGN | sCD299 aa71-399 |
| 10 | sCD301 | MGL/CLEC10A | sCD301 aa61-316 |

This example tests the quantitative binding of intact Spike protein expressed on infectious variants including the B, B.1.5, B.1.1.7, B.1.351, P.1 lineages (BEI Resources, Table 4) to recombinant host receptors, including sACE2, sAPN, sNRP1, sDPP4, sCD33, sCD329, sCD206, sCD209, sCD299, and sCD301 (Table 3). This example focuses on infectious viruses rather than recombinant Spike proteins because post-translational modifications of Spike proteins on infectious virions may differ from post-translational modifications of recombinant Spike proteins produced by HEK or CHO cells. For example, many host receptors recognize specific immature mannose-rich oligosaccharides of the Spike protein glycan shield that would be present on viral surfaces but not on recombinant viral glycoproteins. Selected innate immune receptors have evolved to recognize viral glycoproteins synthesized by virally-infected host cells given that virus-mediated global inhibition of host-cell protein synthesis and overwhelming virus production may block normal maturation of N- and O-linked oligosaccharides. Innate recognition of a perturbed glycan shield is the modus operandi of the CD33, CD329, CD206, CD209, CD299, and CD301 innate receptors. For example, recognition of altered oligosaccharide signatures by innate immune receptors CD206, CD209, CD299, and CD301 on macrophages facilitates phagocytosis and destruction of these viral species. However, some viral pathogens have co-opted these host receptors to infect host cells. Alternatively, viruses may simply dock to the surfaces of highly-mobile leukocytes to aid dissemination of the virus throughout the body to seed secondary and tertiary sites of infection.

TABLE 4
SARS CoV-2 variants from BEI Resources

| BEI # | Lineage | Clade | GISAID ID | Spike Mutations |
|---|---|---|---|---|
| NR-53565 | B | L | EPI_ISL_425177 | No mutations |
| NR-53944 | B.1.5 | G | EPI_ISL_461705 | D614G |
| NR-54011 | B.1.1.7 | GR | EPI_ISL_751801 | HV69-70del, Y144del, N501Y, A570D, |
| | | | | D614G, P681H, T716I, S982A, D1118H |
| NR-54009 | B.1.351 | GH | EPI_ISL_678615 | L18F, D80A, D215G, LAL242-244del, |
| | | | | K417N, E484K, N501Y, D614G, A701V |
| NR-54982 | P.1 | GR | EPI_ISL_792683 | L18F, T20N, P26S, D138Y, R190S, K417T, |
| | | | | E484K, N501Y, D614G, H655Y, T1027I, |
| | | | | V1176F |

### a: Experimental Strategy

Virus pull-down assays are used to quantitatively measure interactions of intact Spike protein on infectious B, B.1.5, B.1.1.7, B.1.351, P.1 viruses with sACE2, sAPN, sNRP1, sDPP4, sCD33, sCD329, sCD206, sCD209, sCD299, or sCD301 recombinant proteins. HCoV 229E, HCoV OC43, HCoV NL63, and H1N1 Influenza (BEI Resources NR-52726, NR-52725, NR-470, NR-29025, respectively) are used as comparative references and controls for these experiments (Table 5). Whole-virus pull-down assays are based on the Twin-Strept-Tag motifs in the C-terminus of the soluble host receptor combined with the use of Strep-Tactin-XT magnetic beads (IBA LifeSciences). Standardized stocks of each virus are produced by infection of VeroE6 cells or other suitable permissive cell lines. Fixed titers of virus stocks are incubated with a concentration range (1 nM to 10 μM) of a soluble host receptor (Table 3). Virus-receptor complexes are purified by use of Strep-Tactin-XT magnetic beads. Virus-receptor-magnetic bead complexes are thoroughly washed to isolate virus-bead complexes. Virus eluted from the Strep-Tactin-XT magnetic beads are subjected to plaque assays to quantitatively measure the amount of infectious virus. Quantitative levels of virus within these complexes are measured by quantitating total protein (from detergent lysates), viral genomic RNA, and viral proteins. Quantification viral RNA are based on RT-qPCR, and quantitation of viral protein are assessed via SDS-PAGE and Western blotting with commercially available reagents.

In whole virus pull-down assays, varying the concentration of the host receptor protein (1 nM to 10 μM) generates sigmoidal binding curves representing the affinity and capacity of virus/host receptor interactions that are captured on magnetic beads. Endpoints reveal no virus if the host receptor does not interact with the virus. Endpoints will reveal abundant virus If the host receptor exhibits high-affinity, high-capacity binding to the virus. Intermediate levels of host receptor binding to virus yield intermediate levels of virus. Concentrations of host receptor that provide half-maximal binding provide a surrogate of affinity/avidity of the host receptor/virus interaction. The plateau (or peak) of the sigmoidal curve provides a surrogate of the capacity/density by which the host receptor saturates the exterior of the virus. The outcome is a matrix with the variants listed in one dimension and the binding of the 10 candidate host receptors listed in another dimension (Table 5). This assay is advantageous because the approach incorporates the native post-translational structure of the variant Spike protein together with the density/avidity of the Spike protein on the surface of infectious variant. The post-translational structure of Spike may be critical due to the unique structural oligosaccharide moieties that decorate the exterior of an infectious virus. Also, the variants may differ in the density of Spike proteins on the virion, which may profoundly affect the avidity of interactions with various accessory host receptor systems. These issues are critical for understanding the virus-host interactions that underlie the pathogenesis of COVID-19. The prediction is that the Spike protein mutations that distinguish the SARS CoV-2 variants confer differential interactions with this set of host receptors, which are interpreted as either qualitative or quantitative differences in the ability of the variant to bind particular host receptors. Because several variant mutations affect binding to ACE2, differences in variant binding of ACE2 compared to differences in variant binding to other host receptors will provide a benchmark for meaningful differences. This analysis broadens the perspective from ACE2 to a more comprehensive view of viral interactions with a range of potential host receptors.

TABLE 5
Predicted positive interactions of viruses
with recombinant soluble host receptors.

| Virus/Variant | sACE2 | sAPN | sNRP1 | sDPP4 | Innate Receptors |
|---|---|---|---|---|---|
| B | Yes | | Yes | | Yes or variable |
| B.1.5 | Yes | | Yes | | Yes or variable |
| B.1.1.7 | Yes | | Yes | | Yes or variable |
| B.1.351 | Yes | | Yes | | Yes or variable |
| P.1 | Yes | | Yes | | Yes or variable |
| HCoV 229E | | Yes | | | Yes or variable |
| HCoV OC43 | | | | | Yes or variable |
| HCoV NL63 | Yes | | | | Yes or variable |
| H1N1 Flu | | | | | Yes or variable |

(Innate Receptors=sCD33, sCD329, sCD206, sCD209, sCD299, sCD301). The predicted outcomes (Table 5) list the predicted qualitative outcomes whereas the true value of the research will reveal quantitative outcomes regarding interactions of the soluble receptors with the given virus. Blank values are unknowns. The assumption is that high avidity interactions of a given host receptor with virus will reflect the potential virological significance. Recombinant host receptor proteins will also be tested in flow cytometric analyses that will measure binding of these soluble host receptors to stably-transfected HEK cells that express SARS CoV-1 Spike, SARS CoV-2 Spike, HCoV-229E Spike, and MERS Spike. HEK-expression of Spike of SARS CoV-1, MERS, 229E, and OC43 were included to provide positive and negative controls and comparative measurements for Spike proteins that bind ACE2, APN, or DPP4. If the variants show no significant differences in the engagement of these host receptors, then this information is nonetheless important in defining a potential monolithic host receptor usage.

New variants can be incorporated/added as new variants become available. There is particular interest in the B.1.429 and B.1.526 variants. B.1.429 (CAL.20C) (S1 mutations S13I, W152C, L452R) is a new variant that emerged in California in which the L452R mutation is believed to foster an immune escape phenotype and/or increased transmissibility (8). The S13I and W152C mutations are concerns in that the S13I mutation may extend the signal peptide to eclipse the 15C aa residue, whereas the W152C mutation introduces an unpaired Cys residue, Loss of 15C and gain of 152C may change the pattern of disulfide linkages and thereby may globally alter the conformation of the N-terminal S1 domain to confer a broad immune escape phenotype. B.1.526 (L5F, T95I, D253G, E484K, S477N, 0614) is a newly emergent lineage in the New York region (20). Both E484K and D253G mutations are believed to confer an immune escape phenotype whereas S477N has been implicated in increased viral infectivity via enhanced affinity for ACE2. Aside from these variants, myriads of additional variants are rapidly evolving in the face of pre-established anti-SARS CoV-2 immunity. Many emerging mutations likely affect interactions with alternative host receptors, which may play important roles in pathogenesis. How these variant Spike domains interface with accessory receptors may be key for unraveling the evolutionary paths that may lead to increased cellular infectivity, systemic viral dissemination within the body, and person-to-person spread across the globe. Given the inconsistencies and gaps in global surveillance, one would expect that myriads of unknown variants are now rapidly evolving below the radar.

Primary priority is given to virus/host receptor interactions that exhibit substantial differences between variants. High priority will also be given to high capacity-high avidity virus/host interactions.

### b: Experimental Strategy (Leukocyte Subsets)

Given that many of these 10 soluble host receptors are expressed as transmembrane receptors on human leukocytes, a critical question pertains to mechanisms by which SARS CoV-2 interfaces with human leukocyte subsets. Assessing the expression of these SARS CoV-2 host receptors on an array of leukocyte subsets is proposed. This sets the stage to assess whether a given host receptor on a given leukocyte subset supports infection by a given SARS CoV-2 variant.

TABLE 6
Flow cytometric analyses of host receptors
(Table 3) on human leukocyte subsets.
Human Leukocyte Subset Preparation

Vero-E6 cells (plus control lines HEK-ACE2, HEK-DPP4, HEK-APN)
Unstimulated fresh PBMCs or derivative lines (below)
Primary macrophage lines: MCSF ± IL-4 ± IL-13 (7-day
cultures) last 48 hrs ± IFN-γ
Primary dendritic cell lines: GM-CSF ± IL-4 ± IL-13
(7-day cultures) last 24-48 hrs ± LPS
Primary human plasmacytoid DCs isolated via positive selection
(BDCA-4/NRP1 beads)
Sorted CD4 T cells activated 3-days with anti-CD3/CD28 mAbs
(Th1, Th2, Th17, Treg)
Sorted CD4 T cells activated 7-days in Mixed Lymphocyte Reaction
(Th1, Th2, Th17, Treg)
Sorted CD4 T cells activated 7-days in Mixed Lymphocyte Reaction
(IFN-β or TGF-β) ± sCD25
Short-term and long-term lines of CD4⁺ or CD8⁺ FOXP3⁺
regulatory T cells
Sorted CD8 T cells or NK cells activated ± IL-2 ± IFN-γ
Sorted CD19⁺ B cells activated 7-days with anti-IgM F(ab)₂ +
IL-4 ± soluble multimeric BAFF

The selection of human PBMCs and differentiated leukocytic cells is designed to comprehensively represent the major myeloid and lymphoid subsets that may have functionally-relevant interactions with a SARS CoV-2 variant. Multicolor gateway mAb panels will include CD45 (pan-leukocytes), CD3, CD4, CD8, TCRγδ, CD25, CD19, CD38, HLA-DR, CD16, CD14, CD11b, CD11c, and TMPRSS2 (which is needed to activate the Spike fusion machinery). Specialized multicolor mAb panels are used for specialized lineages, which will include intracellular staining of cytokines and subset-specific transcription factors (RORC, T-bet, GATA3, FOXP3, etc.). The strategy employs commercial fluorochrome-conjugated mAbs against the 10 host receptors to map expression of the major host receptors to major subsets of naïve/activated, immature/mature leukocytes, including major myeloid and lymphoid lineages (Table 6). Myeloid populations will include monocytes, resting macrophages, activated M1 or M2-polarized macrophages, and granulocytes. DCs will include immature and mature DCs and both conventional and plasmacytoid lineages. Lymphoid populations will include rested and activated CD4⁺ T cells differentiated into the T-bet⁺ Th1, GATA3⁺ Th2, RORC⁺ Th17, and FOXP3⁺ Treg lineages by standard approaches. Regulatory T cell subsets will include continuous lines of IFN-β-induced FOXP3⁺ Tregs and TGF-β-induced FOXP3⁺ Tregs derived in the context of low-zone IL-2 signaling with soluble CD25 (sCD25) (21-23). CD8 T cells will include naïve T cells and rested or activated memory/effector CD8⁺ subsets. Lymphoid cells will include NK cells, naïve B cells, and short-term lines of plasmablasts. Short-term lines (3-7 days) as well as continuous lines (≥10 days) are used to assess the stability of host receptor expression.

### c: Experimental Strategy (Function)

For biologically/statistically significant binding of variants to particular host receptors (a.) and abundant expression of those particular host receptors on a given leukocyte subset (b.), we propose to assess whether that selected set of variants (i.e., those showing differences in host receptor usage or high-capacity binding) productively engages those host-receptor⁺ leukocyte subsets. We will also assess the presence or absence of productive infection. Priority is given to leukocyte subsets that express TMPRSS2, given the role of this protease in productive infection. Infection of VeroE6 cells serves as a positive control. VeroE6 cells or relevant leukocytes are plated overnight in 96-well plates, and then these cells are incubated with the infectious virus (0.032 to 3.2 MOI, half-log increments) for 1 hr. Cells are washed to remove free virus and incubated for 18-24 hrs. Cell lysates are collected for analysis by RT-qPCR via a CFX96 Touch Real-Time PCR System (Bio-Rad). For meaningful virus—host cell interactions, soluble host receptors (Table 3) are used in blocking experiments to assess the identity of the host receptor that enables the virus-host interaction.

This example provides the foundation for testing whether new Spike mutations affect interactions of the SARS CoV-2 Spike protein with ‘noncanonical’ host receptors of the human immune system. Measurements are conducted based on triplicate samples, and experiments are repeated three times to ensure reproducibility of results. Statistical analysis includes Student t-tests (2 groups) or ANOVA (≥3 groups). A p value <0.05 is considered significant.

### Bibliography & References Cited, Example 2

1. Davies N G, Abbott S, Barnard R C, Jarvis C I, Kucharski A J, Munday J D, Pearson C A B, Russell T W, Tully D C, Washburne A D, Wenseleers T, Gimma A, Waites W, Wong K L M, van Zandvoort K, Silverman J D, Diaz-Ordaz K, Keogh R, Eggo R M, Funk S, Jit M, Atkins K E, Edmunds W J. Estimated transmissibility and impact of SARS-CoV-2 lineage B.1.1.7 in England. Science (2021). Epub 2021/03/05. doi: 10.1126/science.abg3055. PubMed PMID: 33658326.
2. Davies N G, Jarvis C I, Edmunds W J, Jewell N P, Diaz-Ordaz K, Keogh R H. Increased mortality in community-tested cases of SARS-CoV-2 lineage B.1.1.7. Nature (2021). Epub 2021/03/17. doi: 10.1038/s41586-021-03426-1. PubMed PMID: 33723411.
3. Galloway S E, Paul P, MacCannell D R, Johansson M A, Brooks J T, MacNeil A, Slayton R B, Tong S, Silk B J, Armstrong G L, Biggerstaff M, Dugan V G. Emergence of SARS-CoV-2 B.1.1.7 Lineage—United States, Dec. 29, 2020-Jan. 12, 2021. MMWR Morb Mortal Wkly Rep (2021) 70(3):95-9. Epub 2021/01/22. doi: 10.15585/mmwr.mm7003e2. PubMed PMID: 33476315; PubMed Central PMCID: PMCPMC7821772 Journal Editors form for disclosure of potential conflicts of interest. No potential conflicts of interest were disclosed.
4. Rahimi F, Talebi Bezmin Abadi A. Implications of the Emergence of a New Variant of SARS-CoV-2, VUI-202012/01. Arch Med Res (2021). Epub 2021/02/03. doi: 10.1016/j.arcmed.2021.01.001. PubMed PMID: 33526352; PubMed Central PMCID: PMCPMC7826083.
5. Muñoz M, Patiño L H, Ballesteros N, Paniz-Mondolfi A, Ramírez J D. Characterizing SARS-CoV-2 genome diversity circulating in South American countries: signatures of potentially emergent lineages? Int J Infect Dis (2021). Epub 2021/02/23. doi: 10.1016/j.ijid.2021.02.073. PubMed PMID: 33618008; PubMed Central PMCID: PMCPMC7895695.
6. Mwenda M, Saasa N, Sinyange N, Busby G, Chipimo P J, Hendry J, Kapona O, Yingst S, Hines J Z, Minchella P, Simulundu E, Changula K, Nalubamba K S, Sawa H, Kajihara M, Yamagishi J, Kapin'a M, Kapata N, Fwoloshi S, Zulu P, Mulenga L B, Agolory S, Mukonka V, Bridges D J. Detection of B.1.351 SARS-CoV-2 Variant Strain—Zambia, December 2020. MMWR Morb Mortal Wkly Rep (2021) 70(8):280-2. Epub 2021/02/26. doi: 10.15585/mmwr.mm7008e2. PubMed PMID: 33630820.
7. Madhi S A, Baillie V, Cutland C L, Voysey M, Koen A L, Fairlie L, Padayachee S D, Dheda K, Barnabas S L, Bhorat Q E, Briner C, Kwatra G, Ahmed K, Aley P, Bhikha S, Bhiman J N, Bhorat A E, du Plessis J, Esmail A, Groenewald M, Home E, Hwa S H, Jose A, Lambe T, Laubscher M, Malahleha M, Masenya M, Masilela M, McKenzie S, Molapo K, Moultrie A, Oelofse S, Patel F, Pillay S, Rhead S, Rodel H, Rossouw L, Taoushanis C, Tegally H, Thombrayil A, van Eck S, Wibmer C K, Durham N M, Kelly E J, Villafana T L, Gilbert S, Pollard A J, de Oliveira T, Moore P L, Sigal A, Izu A. Efficacy of the ChAdOx1 nCoV-19 Covid-19 Vaccine against the B.1.351 Variant. N Engl J Med (2021). Epub 2021/03/17. doi: 10.1056/NEJMoa2102214. PubMed PMID: 33725432.
8. Starr T N, Greaney A J, Dingens A S, Bloom J D. Complete map of SARS-CoV-2 RBD mutations that escape the monoclonal antibody LY-CoV555 and its cocktail with LY-CoV016. bioRxiv (2021). Epub 2021/03/04. doi: 10.1101/2021.02.17.431683. PubMed PMID: 33655250; PubMed Central PMCID: PMCPMC7924270.
9. Ramanathan M, Ferguson I D, Miao W, Khavari P A. SARS-CoV-2 B.1.1.7 and B.1.351 Spike variants bind human ACE2 with increased affinity. bioRxiv (2021). Epub 2021/03/04. doi: 10.1101/2021.02.22.432359. PubMed PMID: 33655251; PubMed Central PMCID: PMCPMC7924271.
10. Martin D P, Weaver S, Tegally H, San E J, Shank S D, Wilkinson E, Giandhari J, Naidoo S, Pillay Y, Singh L, Lessells R J, Gupta R K, Wertheim J O, Nekturenko A, Murrell B, Harkins G W, Lemey P, MacLean O A, Robertson D L, de Oliveira T, Pond S L K. The emergence and ongoing convergent evolution of the N501Y lineages coincides with a major global shift in the SARS-CoV-2 selective landscape. medRxiv (2021). Epub 2021/03/11. doi: 10.1101/2021.02.23.21252268. PubMed PMID: 33688681; PubMed Central PMCID: PMCPMC7941658.
11. Faria N R, Mellan T A, Whittaker C, Claro I M, Candido D D S, Mishra S, Crispim M A E, Sales F C, Hawryluk I, McCrone J T, Hulswit R J G, Franco L A M, Ramundo M S, de Jesus J G, Andrade P S, Coletti T M, Ferreira G M, Silva C A M, Manuli E R, Pereira R H M, Peixoto P S, Kraemer M U, Gaburo N, Camilo C D C, Hoeltgebaum H, Souza W M, Rocha E C, de Souza L M, de Pinho M C, Araujo L J T, Malta F S V, de Lima A B, Silva J D P, Zauli D A G, Ferreira A C S, Schnekenberg R P, Laydon D J, Walker P G T, Schlüter H M, Dos Santos A L P, Vidal M S, Del Caro V S, Filho R M F, Dos Santos H M, Aguiar R S, Modena J L P, Nelson B, Hay J A, Monod M, Miscouridou X, Coupland H, Sonabend R, Vollmer M, Gandy A, Suchard M A, Bowden T A, Pond S L K, Wu C H, Ratmann O, Ferguson N M, Dye C, Loman N J, Lemey P, Rambaut A, Fraiji N A, Carvalho M, Pybus O G, Flaxman S, Bhatt S, Sabino E C. Genomics and epidemiology of a novel SARS-CoV-2 lineage in Manaus, Brazil. medRxiv (2021). Epub 2021/03/11. doi: 10.1101/2021.02.26.21252554. PubMed PMID: 33688664; PubMed Central PMCID: PMCPMC7941639.
12. Firestone M J, Lorentz A J, Meyer S, Wang X, Como-Sabetti K, Vetter S, Smith K, Holzbauer S, Beaudoin A, Garfin J, Ehresmann K, Danila R, Lynfield R. First Identified Cases of SARS-CoV-2 Variant P.1 in the United States—Minnesota, January 2021. MMWR Morb Mortal Wkly Rep (2021) 70(10):346-7. Epub 2021/03/12. doi: 10.15585/mmwr.mm7010e1. PubMed PMID: 33705367.
13. Wang P, Wang M, Yu J, Cerutti G, Nair M S, Huang Y, Kwong P D, Shapiro L, Ho D D. Increased Resistance of SARS-CoV-2 Variant P.1 to Antibody Neutralization. bioRxiv (2021). Epub 2021/03/11. doi: 10.1101/2021.03.01.433466. PubMed PMID: 33688656; PubMed Central PMCID: PMCPMC7941628 provisional patent application on mAbs to SARS-CoV-2.
14. Okada P, Buathong R, Phuygun S, Thanadachakul T, Parnmen S, Wongboot W, Waicharoen S, Wacharapluesadee S, Uttayamakul S, Vachiraphan A, Chittaganpitch M, Mekha N, Janejai N, lamsirithaworn S, Lee R T, Maurer-Stroh S. Early transmission patterns of coronavirus disease 2019 (COVID-19) in travellers from Wuhan to Thailand, January 2020. Euro Surveill (2020) 25(8). Epub 2020/03/05. doi: 10.2807/1560-7917.Es.2020.25.8.2000097. PubMed PMID: 32127124; PubMed Central PMCID: PMCPMC7055038.
15. Groves D C, Rowland-Jones S L, Angyal A. The D614G mutations in the SARS-CoV-2 spike protein: Implications for viral infectivity, disease severity and vaccine design. Biochem Biophys Res Commun (2020). Epub 2020/11/18. doi: 10.1016/j.bbrc.2020.10.109. PubMed PMID: 33199022; PubMed Central PMCID: PMCPMC7643658.
16. Jackson C B, Zhang L, Farzan M, Choe H. Functional importance of the D614G mutation in the SARS-CoV-2 spike protein. Biochem Biophys Res Commun (2020). Epub 2020/11/23. doi: 10.1016/j.bbrc.2020.11.026. PubMed PMID: 33220921; PubMed Central PMCID: PMCPMC7664360.
17. Korber B, Fischer W M, Gnanakaran S, Yoon H, Theiler J, Abfalterer W, Hengartner N, Giorgi E E, Bhattacharya T, Foley B, Hastie K M, Parker M D, Partridge D G, Evans C M, Freeman T M, de Silva T I, McDanal C, Perez L G, Tang H, Moon-Walker A, Whelan S P, LaBranche C C, Saphire E O, Montefiori D C. Tracking Changes in SARS-CoV-2 Spike: Evidence that D614G Increases Infectivity of the COVID-19 Virus. Cell (2020) 182(4):812-27.e19. Epub 2020/07/23. doi: 10.1016/j.cell.2020.06.043. PubMed PMID: 32697968; PubMed Central PMCID: PMCPMC7332439.
18. Plante J A, Liu Y, Liu J, Xia H, Johnson B A, Lokugamage K G, Zhang X, Muruato A E, Zou J, Fontes-Garfias C R, Mirchandani D, Scharton D, Bilello J P, Ku Z, An Z, Kalveram B, Freiberg A N, Menachery V D, Xie X, Plante K S, Weaver S C, Shi P Y. Spike mutation D614G alters SARS-CoV-2 fitness and neutralization susceptibility. bioRxiv (2020). Epub 2020/09/11. doi: 10.1101/2020.09.01.278689. PubMed PMID: 32908978; PubMed Central PMCID: PMCPMC7480025 reverse genetic system and reporter SARS-CoV-2. Other authors declare no competing interests.
19. Zhang L, Jackson C B, Mou H, Ojha A, Rangarajan E S, Izard T, Farzan M, Choe H. The D614G mutation in the SARS-CoV-2 spike protein reduces S1 shedding and increases infectivity. bioRxiv (2020). Epub 2020/06/27. doi: 10.1101/2020.06.12.148726. PubMed PMID: 32587973; PubMed Central PMCID: PMCPMC7310631.
20. Annavajhala M K, Mohri H, Zucker J E, Sheng Z, Wang P, Gomez-Simmonds A, Ho D D, Uhlemann A C. A Novel SARS-CoV-2 Variant of Concern, B.1.526, Identified in New York. medRxiv (2021). Epub 2021/03/04. doi: 10.1101/2021.02.23.21252259. PubMed PMID: 33655278; PubMed Central PMCID: PMCPMC7924303.
21. DeOca K B, Moorman C D, Garcia B L, Mannie M D. Low-Zone IL-2 Signaling: Fusion Proteins Containing Linked CD25 and IL-2 Domains Sustain Tolerogenic Vaccination in vivo and Promote Dominance of FOXP3(+) Tregs in vitro. Front Immunol (2020) 11:541619. Epub 2020/10/20. doi: 10.3389/fimmu.2020.541619. PubMed PMID: 33072087; PubMed Central PMCID: PMCPMC7538601.
22. Wilkinson D S, Ghosh D, Nickle R A, Moorman C D, Mannie M D. Partial CD25 antagonism enables dominance of antigen-inducible CD25high FOXP3+ regulatory T cells as a basis for a Treg-based adoptive immunotherapy. Front Immunol (2017) https://doi.org/10.3389/fimmu.2017.01782. PubMed PMID: 29312311; PubMed Central PMCID: PMC5735073.
23. Wang D, Ghosh D, Islam S M, Moorman C D, Thomason A E, Wilkinson D S, Mannie M D. IFN-beta Facilitates Neuroantigen-Dependent Induction of CD25+ FOXP3+ Regulatory T Cells That Suppress Experimental Autoimmune Encephalomyelitis. J Immunol (2016) 197:2992-3007. Epub 2016/09/14. doi: 10.4049/jimmunol.1500411. PubMed PMID: 27619998; PubMed Central PMCID: PMC5101178.

The foregoing is illustrative of the present inventive concept and is not to be construed as limiting thereof. Further embodiments of the present inventive concept are exemplified in the following claims.

1. A fusion polypeptide comprising:
a pathogen recognition domain;
a linker region; and
an interferon domain.

2. The fusion polypeptide of claim 1, wherein the pathogen recognition domain comprises an angiotensin-converting enzyme-2 (ACE2) domain.

3. The fusion polypeptide of claim 2, wherein the ACE2 domain is a soluble form of the ACE2 domain.

4. The fusion polypeptide of claim 3, wherein the soluble form of the ACE2 domain comprises a truncated form of the soluble form of the ACE2 domain.

5. The fusion polypeptide of claim 2, wherein the ACE2 domain is enzymatically inactive.

6. The fusion polypeptide of claim 1, wherein the pathogen recognition domain comprises an APN, NRP1, DPP4, CD33 (SIGLEC-3), CD329 (SIGLEC-9), CD206 (MMR), CD209 (DC-SIGN), CD299 (L-SIGN), or a CD301 domain.

7. The fusion polypeptide of claim 1, wherein the pathogen recognition domain is an antibody or antibody fragment domain.

8. The fusion polypeptide of claim 1, wherein the interferon domain is a Type I, Type II, or Type III interferon domain.

9. The fusion polypeptide of claim 1, wherein the interferon domain is a human interferon domain.

10. The fusion polypeptide of claim 9, wherein the human interferon domain is a human IFN-β domain.

11. The fusion polypeptide of claim 1, wherein the N-terminal of the fusion polypeptide comprises the pathogen recognition domain.

12. The fusion polypeptide of claim 1, wherein the N-terminal of the fusion polypeptide comprises the interferon domain.

13. The fusion polypeptide of claim 1, wherein the pathogen recognition domain binds to a viral surface antigen.

14. The fusion polypeptide of claim 13, wherein the viral surface antigen is a SARS2 surface antigen.

15. The fusion polypeptide of claim 13, wherein viral surface antigen is a SARS2 spike (S) protein.

16. A pharmaceutical composition comprising the fusion polypeptide of claim 1.

17-29. (canceled)

30. A method of treating a subject in need thereof comprising administering a therapeutically effective amount of the fusion polypeptide of claim 1.

31-40. (canceled)

41. A method of treating a viral infection comprising administering a therapeutically effective amount of the fusion polypeptide of claim 1 to a subject in need thereof.

42. (canceled)

43. The method of claim 41, wherein the viral infection comprises an infection with a Coronaviridae family virus.

44-48. (canceled)

49. The method of claim 41, wherein the viral infection comprises an infection with an Influenza virus or an infection with HIV.

50. (canceled)


SEQUENCE LISTING (8 sequences)

SEQ ID NO: 1 - protein, 5 aa, Artificial
  Fusion protein linker sequence
GGGGS

SEQ ID NO: 2 - protein, 20 aa, Artificial
  Fusion protein linker sequence
GGGGSGGGGSGGGGSGGGGS

SEQ ID NO: 3 - protein, 25 aa, Artificial
  Fusion protein linker sequence
GGGGSGGGGSGGGGSGGGGSGGGGS

SEQ ID NO: 4 - protein, 29 aa, Artificial
  Fusion protein linker sequence
GGGGSTRGGGGSTHHHHHHHHHTSGGGGS

SEQ ID NO: 5 - protein, 5 aa, Artificial
  Fusion protein linker sequence
DDDDK

SEQ ID NO: 6 - protein, 16 aa, Artificial
  Fusion protein linker sequence
GDDDDKGHHHHHHHHH

SEQ ID NO: 7 - protein, 17 aa, Artificial
  Fusion protein linker sequence
AKGGGSEGGGSEGGGSG

SEQ ID NO: 8 - protein, 7 aa, Artificial
  Fusion protein linker sequence
GDDDDKG